(19) United States
(12) Patent Application Publication
(10) Pub. No.: US 2012/0238882 A1
(43) Pub. Date: Sep. 20, 2012

(54) SKIN OPTICAL DIAGNOSING APPARATUS AND OPERATING METHOD THEREOF

(76) Inventors: Chung-Cheng CHOU, Taoyuan County (TW); William WANG, Taoyuan City (TW); Chung-Ping CHUANG, Taoyuan City (TW)

(21) Appl. No.: 13/421,214

(22) Filed: Mar. 15, 2012

(30) Foreign Application Priority Data
Mar. 15, 2011 (TW) 100108754

(51) Int. Cl. A61B 6/00

(52) U.S. Cl. 600/476

(57) ABSTRACT

A skin optical diagnosing apparatus and operating method thereof are disclosed. The skin optical diagnosing apparatus includes a positioning module, an optical sensing module, a processing module, and a display module. The skin optical diagnosing apparatus uses the positioning module to select a target region on a sample, and then the optical sensing module performs an optical sensing on the target region to obtain an optical information data related to the target region. The processing module is used for processing the optical information data to generate an optical diagnosed result. The display module is used for displaying the optical diagnosed result.

### BACKGROUND OF THE INVENTION

1. Field of the Invention

The invention relates to optical diagnosing; in particular, to a skin optical diagnosing apparatus and operating method thereof with the function of confirming diagnosed positions.

2. Description of the Prior Art

In recent years, with the continuous development of medical technology and biotechnology, the regions of medical diagnosing and biochemical test have become more and more important. Therefore, various instruments related to medical diagnosing and biochemical test are shown in the market. Especially, beauty care is more and more popular is the modern society; the skin test equipment used to test the state of human body skin can attract the attention of consumers.

In general, the conventional skin test equipment mainly provides the information related to skin surface state, for example, the information about the size of pores on the skin surface and whether the spots existed on the skin surface. In recent years, the skin test equipment which can test the state of skin underlying tissue has been developed, such as electrode-type or optical-type skin test equipment. Wherein, the former can provide electrical message through the electrode structure for testing skin humidity; the latter can provide image information about the state of skin underlying tissue through the optical structure to be an important reference when the doctor evaluates the body tissue health state of patients.

However, no matter which one of the above-mentioned skin test equipments is used, in practical applications, there is still a serious drawback existed that when the conventional skin test equipment tests on human body skin many times, it is hard to accurately determine that the test positions in every tests are the same, and the doctor is also hard to trace and compare the lesions of the patient.

Therefore, the invention provides a skin optical diagnosing apparatus and operating method thereof to solve the above-mentioned problems occurred in the prior arts.

### SUMMARY OF THE INVENTION

A first embodiment of the invention is a skin optical diagnosing apparatus. In this embodiment, the skin optical diagnosing apparatus includes a positioning module, an optical sensing module, a processing module, and a display module.

Wherein, the positioning module is used for selecting a target region on a sample; the optical sensing module is used for performing an optical sensing on the target region to obtain an optical information data related to the target region; the processing module is used for processing the optical information data to generate an optical diagnosed result; the display module is used for displaying the optical diagnosed result.

In practical applications, the optical sensing module can include at least one optical component, a replaceable adapter interface, and a contact end replacing component. The optical sensing module uses an optical interference technology to perform the optical sensing on a tissue under the target region of the sample to obtain the optical information data related to a longitudinal profile of the tissue.

In addition, the positioning module can have different types to select the target region on the sample through different positioning mechanisms respectively. In an embodiment, the positioning module can include a light receiving unit and a judging unit. The light receiving unit is used for receiving a reflected light formed by a region on the sample reflecting an incident light to generate a reflection result; the judging unit is used for judging whether a difference between the reflection result and a previous reflection result is smaller than a default value, if the judged result of the judging unit is yes, the judging unit determines that the region is the target region.

In another embodiment, the positioning module can include an image capturing unit and an image comparing unit. The image capturing unit is used for capturing an image of a region on the sample; the image comparing unit is used for comparing whether a plurality of features of the image is the same with those of a previous image, if the compared result of the image comparing unit is yes, the image comparing unit determines that the region is the target region.

A second embodiment of the invention is a skin optical diagnosing apparatus operating method. In this embodiment, the kin optical diagnosing apparatus operating method is used to operate a skin optical diagnosing apparatus. The skin optical diagnosing apparatus includes a positioning module, an optical sensing module, a processing module, and a display module.

The skin optical diagnosing apparatus operating method includes steps of: (a) the positioning module selecting a target region on a sample; (b) the optical sensing module performing an optical sensing on the target region to obtain an optical information data related to the target region; (c) the processing module processing the optical information data to generate an optical diagnosed result; (d) the display module displaying the optical diagnosed result.

Compared to prior arts, the skin optical diagnosing apparatus and operating method thereof of the invention can accurately confirm the target test position on the sample is the same with the previous test position through optical positioning method before performing optical sensing on the skin or similar tissues. Therefore, even though the skin optical diagnosing apparatus performs several times of optical diagnosing on human body skin, the positions in every time of optical diagnosing will be the same, so that the doctor can easily trace and compare the lesions of the patient to effectively enhance the quality and the efficiency of medical treatment.

In addition, the optical sensing module of the skin optical diagnosing apparatus in this invention can be designed to be single component type or two kits combination type, and can include a replaceable adapter interface and a contact end replacing component, therefore, it has advantages of easy to upgrade, flexibly designed, avoiding pollution, and proving personal usage. If the optical sensing module is designed to be fixed type, it can cooperate with special rulers to perform more detailed and accurate positioning and comparing, and the optical sensing module can be also integrated with the positioning module in the same structure to perform large-area scanning test, so that the data of large-area preliminary observation, small-area detailed test, and test path record can be established respectively to help the doctor to continuously observe and trance the state of the patient.

The advantage and spirit of the invention may be understood by the following detailed descriptions together with the appended drawings.

### BRIEF DESCRIPTION OF THE APPENDED DRAWINGS

FIG. 1 illustrates a function block diagram of the skin optical diagnosing apparatus in the first embodiment of the invention.

FIG. 2A illustrates a function block diagram of the positioning module using the light emission/reflection positioning mechanism; FIG. 2B and FIG. 2C illustrate a schematic diagram of the positioning module of the skin optical diagnosing apparatus using the light emission/reflection positioning mechanism to position the target region.

FIG. 3A illustrates a function block diagram of the positioning module using the image comparing and positioning mechanism; FIG. 3B and FIG. 3C illustrate a schematic diagram of the positioning module of the skin optical diagnosing apparatus using the image comparing and positioning mechanism to position the target region; FIG. 3D illustrate a schematic diagram of the image of the surface of the sample; FIG. 3E illustrate a schematic diagram of the previous image.

FIG. 4A and FIG. 4B illustrate the optical sensing modules with single component type design and with combination type design of two sets of kits respectively.

FIG. 5 illustrates a schematic diagram of the optical sensing module cooperating with special ruler to perform positioning and comparing on the region of the sample.

FIG. 6 illustrates a schematic diagram of the optical sensing module and the display module integrated in the structure of the notebook.

FIG. 7A illustrates a schematic diagram of the skin optical diagnosing apparatus in the prior art maintaining the fixed moving path to perform the optical sensing process.

FIG. 7B and FIG. 7C illustrate schematic diagrams of the skin optical diagnosing apparatus dynamically adjusting its moving path in response to the ups and downs of the sample.

FIG. 8 illustrates a flowchart of the skin optical diagnosing apparatus operating method of the second embodiment of the invention.

FIG. 9 illustrates a flowchart of the step S100˜S106 of the step S10 in FIG. 8.

FIG. 10 illustrates a flowchart of the step S100′˜S106′ of the step S10 in FIG. 8.

### DETAILED DESCRIPTION OF THE INVENTION

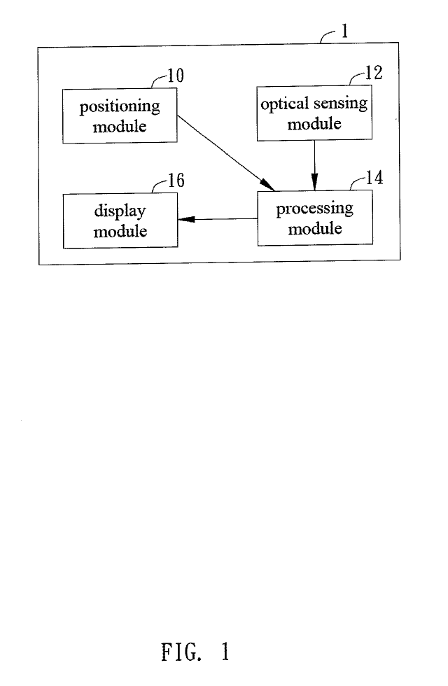

A first embodiment of the invention is a skin optical diagnosing apparatus. In this embodiment, the skin optical diagnosing apparatus is used to perform optical sensing process on a sample, and then perform an early disease diagnosis on the sample according to the optical sensing result. In fact, the sample can be skin or similar tissue without any specific limitations. Please refer to FIG. 1. FIG. 1 illustrates a function block diagram of the skin optical diagnosing apparatus in this embodiment.

As shown in FIG. 1, the skin optical diagnosing apparatus 1 includes a positioning module 10, an optical sensing module 12, a processing module 14, and a display module 16. Wherein, the processing module 14 is coupled to the positioning module 10; the processing module 14 is coupled to the optical sensing module 12; the display module 16 is coupled to the processing module 14. It should be noticed that the positioning module 10 can be coupled to the display module 16 through the processing module 14 as shown in FIG. 1 or directly coupled to the display module 16. That is to say, the display module 16 can be switched to only display the surface of the sample (for confirming diagnosed position) or display the actual longitudinal profile of the sample.

In this embodiment, the modules of the skin optical diagnosing apparatus 1 have following functions respectively: the positioning module 10 is used for selecting a target region on a sample; the optical sensing module 12 is used for performing an optical sensing on the target region to obtain an optical information data related to the target region; the processing module 14 is used for processing the optical information data to generate an optical diagnosed result; the display module 16 is used for displaying the optical diagnosed result.

Next, the modules of the skin optical diagnosing apparatus 1 will be introduced in detail respectively as follows.

In this embodiment, the positioning module 10 can use different positioning mechanisms to select a target region on the sample. For example, the positioning module 10 of the skin optical diagnosing apparatus 1 can use the light emission/reflection positioning mechanism or the image comparing and positioning mechanism to perform the positioning of the target region. The light emission/reflection positioning mechanism has advantage of low cost, and the image comparing positioning mechanism can provide more accurate judgment result. In addition, the positioning module 10 can also use electronic positioning mechanism or other positioning mechanism without specific limitations.

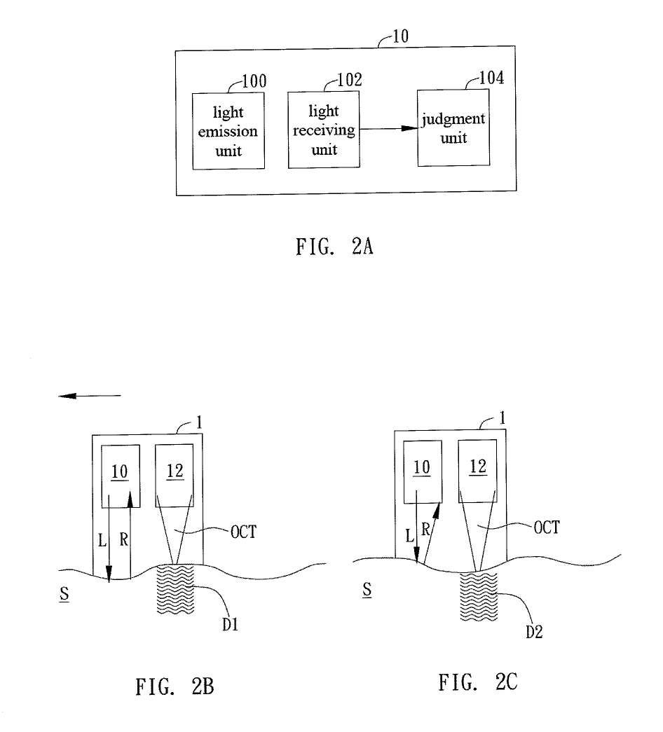

Please refer to FIG. 2A through FIG. 2C. FIG. 2A illustrates a function block diagram of the positioning module 10 using the light emission/reflection positioning mechanism; FIG. 2B and FIG. 2C illustrate a schematic diagram of the positioning module 10 of the skin optical diagnosing apparatus 1 using the light emission/reflection positioning mechanism to position the target region. As shown in FIG. 2A, the positioning module 10 includes a light emission unit 100, a light receiving unit 102, and a judgment unit 104. Wherein, the light receiving unit 102 is coupled to the judgment unit 104.

Please refer to FIG. 2A and FIG. 2B at the same time. The positioning module 10 of the skin optical diagnosing apparatus 1 uses its light emission unit 100 to emit an incident light L to the surface of the sample S, or splits original light to obtain the incident light L. Therefore, the light emission unit 100 is not an indispensable component of the positioning module 10. Then, the positioning module 10 will receive the reflected light R formed by the surface of the sample S reflecting the incident light L through its light receiving unit 102 to generate a reflection result.

Afterward, the positioning module 10 will judge whether a difference between the reflection result and a previous reflection result is smaller than a default value through its judging unit 104. If the judged result of the judging unit 104 is yes, the similarity between the reflection result of the surface of the sample S and the previous reflection result is quite high, therefore, the judging unit 104 will determine that the tested region D1 on the surface of the sample is the target region, and the optical sensing module 12 can emit an optical coherence tomography light to the tested region D1 to perform optical sensing.

If the judged result of the judging unit 104 is no, the similarity between the reflection result of the surface of the sample S and the previous reflection result is not high enough, therefore, the judging unit 104 will determine that the tested region D1 on the surface of the sample is not the target region, and the positioning module 10 will move to repeat the above-mentioned positioning processes on another tested region D2 of the surface of the sample S, as shown in FIG. 2C.

It should be noticed that the reflection result and the previous reflection result can be the angle or strength of the reflected light received by the light receiving unit 102 of the positioning module 10, that is to say, the difference between the reflection result and the previous reflection result can be the angle difference or strength difference between the reflected light and the previous reflected light. Then, the judging unit 104 will determine whether the tested region D1 on the surface of the sample S is the target region according to whether the angle difference or strength difference between the reflected light and the previous reflected light is smaller than a default value, but not limited to this.

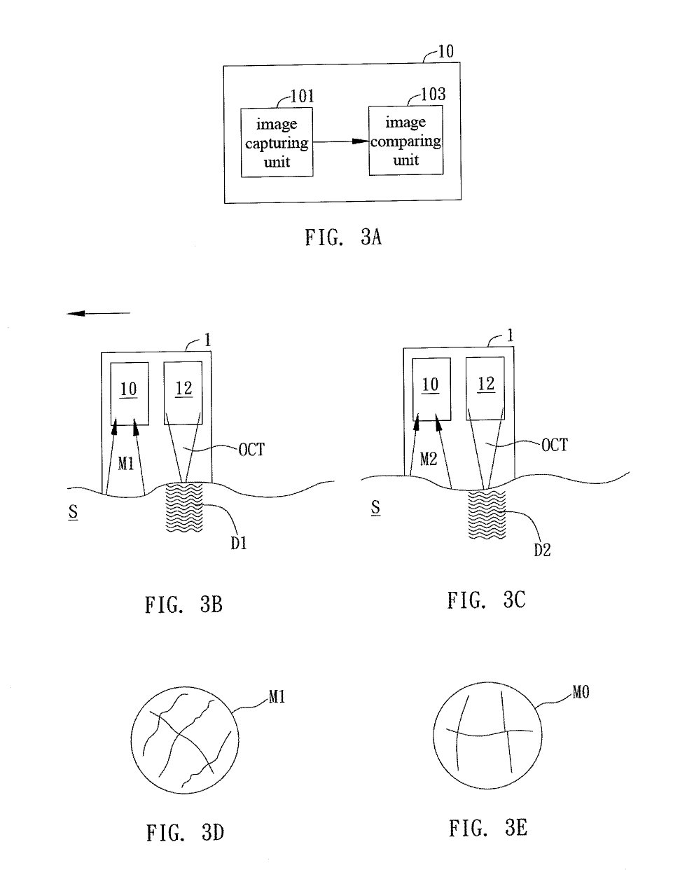

On the other hand, the positioning module 10 can also use the image comparing and positioning mechanism to perform the positioning of the target region. Please refer to FIG. 3A through FIG. 3C. FIG. 3A illustrates a function block diagram of the positioning module 10 using the image comparing and positioning mechanism; FIG. 3B and FIG. 3C illustrate a schematic diagram of the positioning module 10 of the skin optical diagnosing apparatus 1 using the image comparing and positioning mechanism to position the target region. As shown in FIG. 3A, the positioning module 10 includes an image capturing unit 101 and an image comparing unit 103, and the image capturing unit 101 is coupled to the image comparing unit 103.

Please refer to FIG. 3A and FIG. 3B at the same time, the positioning module 10 captures an image M1 of the surface of the sample S through the image capturing unit 101 (as shown in FIG. 3D). Then, the positioning module 10 will compare whether a plurality of features of the image M1 is the same with those of the previous image MO through the image comparing unit 103 (as shown in FIG. 3E). In fact, the above-mentioned features can be the pores, lines, or spots distributed on the surface of the skin, but not limited to this.

If the compared result of the image comparing unit 103 is yes, the similarity between the image M1 and the previous image MO is quite high, therefore, the image comparing unit 103 will determine that the tested region D1 on the surface of the sample is the target region, and the optical sensing module 12 will emit an optical coherence tomography light to the tested region D1 to perform optical sensing. If the compared result of the image comparing unit 103 is no, the similarity between the image M1 and the previous image MO is not high enough, therefore, the image comparing unit 103 will determine that the tested region D1 on the surface of the sample is not the target region, and the positioning module 10 will move to repeat the above-mentioned positioning processes on another tested region D2 of the surface of the sample S, as shown in FIG. 3C.

It should be noticed that the compared subject matter used when the positioning module 10 performs the positioning of the target region is not necessary to be target region itself; it can be also other subject matter around the target region which can let the positioning module 10 correctly position the target region to be optically sensed. Therefore, the compared subject matter used when the positioning module 10 positions is not necessary the same with the target region optically sensed by the optical sensing module 12.

Then, the optical sensing module 12 of the skin optical diagnosing apparatus 1 will be introduced. The optical sensing module 12 of the skin optical diagnosing apparatus 1 in this invention has not specific limitations on its design type. For example, different designs of single component type or combination type can be selected to be used, the built-in or plug-in image sensor can be selected to observe the target region T, or the functions of the optical sensing module 12 and the positioning module 10 are integrated.

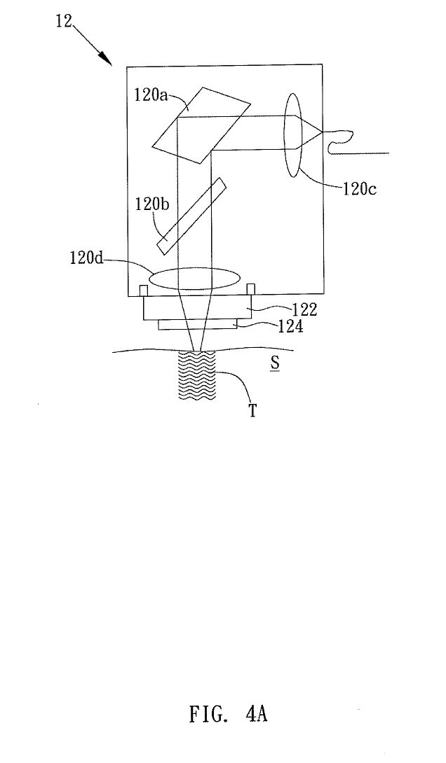

For example, please refer to FIG. 4A and FIG. 4B, FIG. 4A and FIG. 4B illustrate the optical sensing modules 12 with single component type design and with combination type design of two sets of kits 12a and 12b respectively to achieve the effects of flexibly designed, easy to upgrade, and easy to change observation angle.

As shown in FIG. 4A, the optical sensing module 12 can include common optical components (including a rotation mirror 120a, a light splitter 120b, a collimating lens 120c, and an objective lens 120d), a replaceable adapter interface 122, and a contact end replacing component 124. It should be noticed that the light splitter 120b of the optical sensing module 12 can provide the reflected light to the built-in image sensor (not shown in figures), such as CCD or CMOS type of image sensor, but not limited to this. After the splitting light image captured by the image sensor, the splitting light image can be provided to the light splitter 120b, and its main function is that the user can use the reflected light reflected by the surface of the sample S (e.g., the skin) cooperating with the built-in image sensor to observe and position the target region T on the sample S. That is to say, the design of FIG. 4A can be integrated with the positioning module 10.

In FIG. 4B, the optical sensing module 12 is formed by the combination of the two kits 12a and 12b. The kit 12a includes a rotation mirror 120a and a collimating lens 120c; the kit 12b includes an objective lens 120d, a reflector 120e, a replaceable adapter interface 122, a contact end replacing component 124, and a plug-in image sensor 120f. It should be noticed that the optical sensing module 12 in FIG. 4B has no light splitter 120b, therefore, the optical sensing module 12 must include the plug-in image sensor 120f to observe the target region T on the sample S, or to directly determine the target region through the positioning module 10.

In this embodiment, the two reflectors 120e in FIG. 4B can be mirrors, and the distance between the two reflectors 120e has no specific limitations, it can be adjusted according to practical needs. As to the contact end replacing component 124 in FIG. 4A and FIG. 4B, it can be designed to be detachable type, disposable type, or release paper tear-off type to prevent the surface of the contact end of the optical sensing module 12 from being contaminated, and the usage of the optical sensing module 12 can be personalized.

In this embodiment, the optical sensing module 12 can use an optical interference (e.g., an optical coherence tomography technology) to emit the optical coherence tomography light to perform the optical sensing on a tissue under the target region T of the sample S to obtain the optical information data related to a longitudinal profile of the tissue under the target region T. In fact, the depth that the optical sensing module 12 senses the tissue under the target region T can be about 2-3 mm, and the wavelength of the light it uses can be 1300 nm or 840 nm. In order to achieve a goal of rapid sensing, the optical sensing module 12 can use a frequency domain OCT technology to perform optical penetrating sensing on the skin. As to the above-mentioned OCT technology, since it is disclosed to the public in detail, it is not described again here.

The optical information data captured by the optical sensing module 12 is transmitted to the processing module 14 through a light path (e.g., a fiber or a light guide). Then, the processing module 14 will process the received optical information data to analyze the longitudinal profile of skin to generate an optical diagnosed result. At last, the display module 16 will display the optical diagnosed result for the user to observe the optical diagnosed result. In practical applications, there is no specific limitation to the way that the display module 16 displays the optical diagnosed result. For example, the display module 16 can display the optical diagnosed result through images with different colors or shades; the display module 16 can display the optical diagnosed result through the voices with different volumes, frequencies, tempos; the display module 16 can display the optical diagnosed result through different temperatures; the display module 16 can also emit lights with different brightness or colors to display the optical diagnosed result.

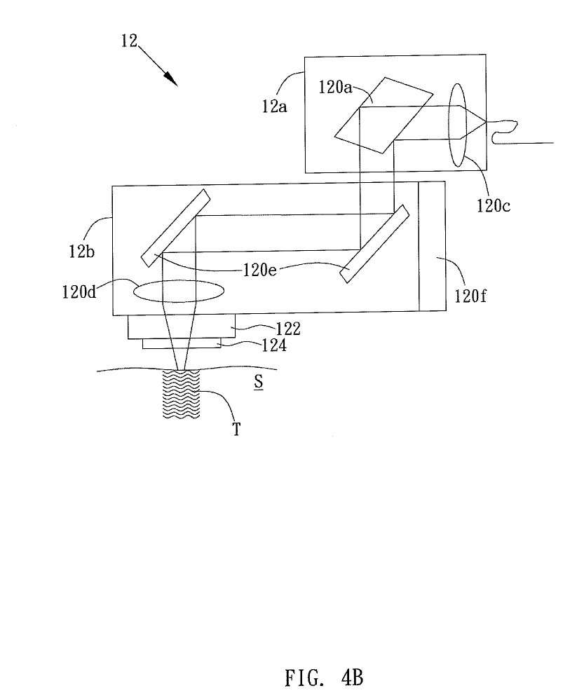

In addition, as shown in FIG. 5, if the optical sensing module 12 is designed as a fixed type, and it can also cooperate with special ruler 20 to perform more detailed and accurate positioning and comparing on the region D of the sample S. Even the portable optical sensing module 12 (including the positioning module 10) can be also integrated with the display module 16 in the same structure (e.g., the computer apparatus 22 such as a notebook, but not limited to this case), as shown in FIG. 6, so that the large-area scanning test can be performed more easily, and the data of large-area preliminary observation, small-area detailed test, and test path record can be established respectively to help the doctor to continuously observe and trance the state of the patient.

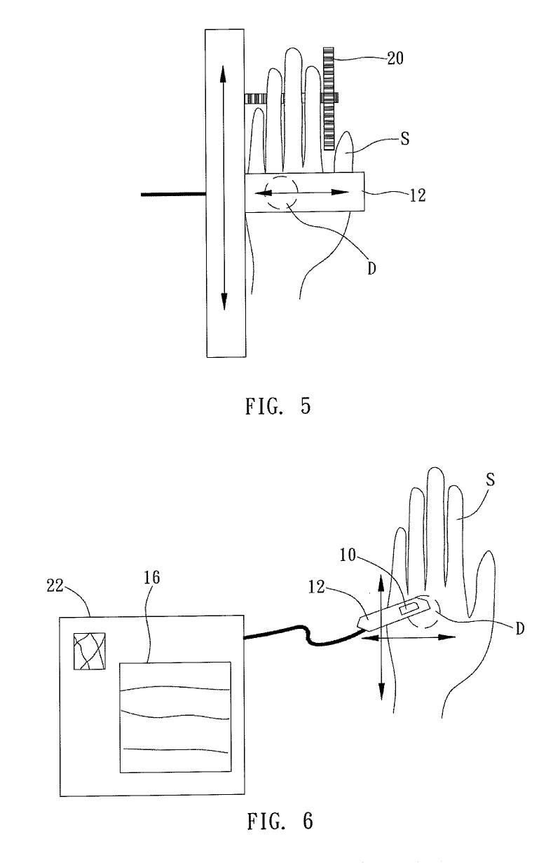

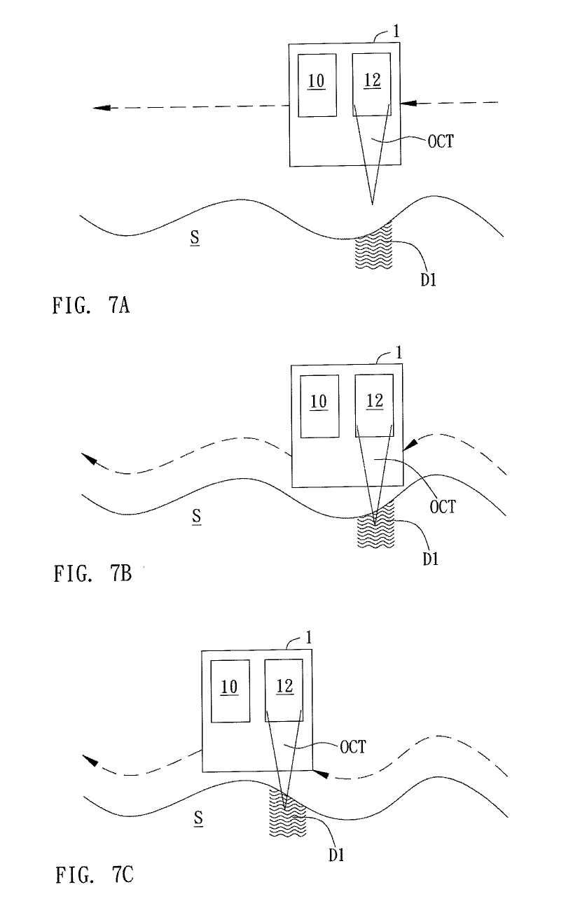

Please refer to FIG. 7A. FIG. 7A illustrates a schematic diagram of the skin optical diagnosing apparatus in the prior art maintaining the fixed moving path to perform the optical sensing process. As shown in FIG. 7A, the skin optical diagnosing apparatus 1 in the prior art maintains the fixed moving path to perform the optical sensing process. Because the sensing depth that the skin optical diagnosing apparatus 1 performs the optical sensing process through the optical sensing module 12 still has some limitations, therefore, if the surface of the sample S (e.g., human's face) is not smooth enough and has larger ups and downs, it is hard for the optical sensing module 12 to accurately perform optical sensing on the target region D1, errors will be easily generated. In fact, this drawback can be improved by closely contacting the optical sensing module 12 with the surface of human's face, however, this pressure caused by closely contacting will make the human's face feel uncomfortable, especially when the surface of human's face has wound on it, it will be pain and the injury will become worse.

In order to solve the above-mentioned problems, the skin optical diagnosing apparatus 1 can effectively perform the optical sensing more accurately on the sample having larger ups and downs in certain ways to avoid errors occurred.

For example, as shown in FIG. 7B and FIG. 7C, the skin optical diagnosing apparatus 1 can dynamically adjust its moving path in response to the ups and downs of the sample S, that is to say, the skin optical diagnosing apparatus 1 can adhere to the surface of the sample S and move along the ups and downs of the sample S (its moving path is the dashed arrow shown in the figure), therefore, the optical sensing module 12 of the skin optical diagnosing apparatus 1 can smoothly perform optical sensing more accurately on the concaved target region D1 and the raised target region D2 of the sample S respectively, and it will not generate errors due to the ups and downs of the sample S as the prior art.

It should be noticed that except the above-mentioned way that the entire skin optical diagnosing apparatus 1 moves along the ups and downs of the sample S, in fact, the skin optical diagnosing apparatus 1 can only move the optical sensing module 12, or only adjust the focal length and depth of field of the optical component in the optical sensing module 12, to perform optical sensing more accurately on the surface of the sample S with larger ups and downs to avoid the errors.

It should be noticed that because the skin optical diagnosing apparatus 1 can be used to diagnose different parts of patient's body, such as the state of the skin of the face or the hand. For convenience, the skin optical diagnosing apparatus 1 can select some feature points or feature regions on different parts of different patient's bodies and record them for the next diagnosis in the future. For example, the feature points or feature regions of patient's face can be eyebrow, eye, lips, nose, distance between two eyes, or length of the philtrum; the feature points or feature regions of patient's hand can be the positions of knuckles or phalanxes of each finger or the distances between them, not limited to this case.

A second embodiment of the invention is a skin optical diagnosing apparatus operating method. In practical applications, the skin optical diagnosing apparatus operating method is applied in a skin optical diagnosing apparatus, but not limited to this. In this embodiment, the skin optical diagnosing apparatus includes a positioning module, an optical sensing module, a processing module, and a display module.

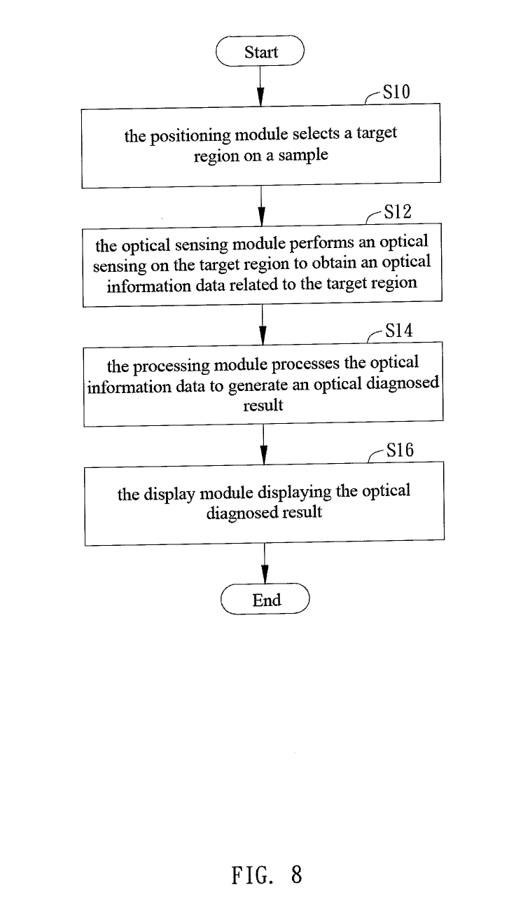

Please refer to FIG. 8. FIG. 8 illustrates a flowchart of the skin optical diagnosing apparatus operating method of this embodiment. As shown in FIG. 8, the skin optical diagnosing apparatus operating method includes the following steps. At first, in step S10, the positioning module selects a target region on a sample.

Then, in step S12, the optical sensing module performs an optical sensing on the target region to obtain an optical information data related to the target region. In detail, the optical sensing module can use an optical interference technology (e.g. an optical coherence tomography (OCT) technology) to perform the optical sensing on a tissue under the target region of the sample to obtain the optical information data related to a longitudinal profile of the tissue, but not limited to this case.

In step S14, the processing module processes the optical information data to generate an optical diagnosed result. Afterward, in step S16, the display module displaying the optical diagnosed result. In fact, the way that the display module displays the optical diagnosed result has no specific limitations. For example, the display module can display the optical diagnosed result through images with different colors or shades, the voices with different volumes, frequencies, tempos, different temperatures, or lights with different brightness or colors.

In practical applications, the positioning module in step S10 can use different positioning mechanisms to select a target region on the sample, such as the light emission/reflection positioning mechanism, the image comparing and positioning mechanism, the electronic positioning mechanism, or other positioning mechanism without specific limitations.

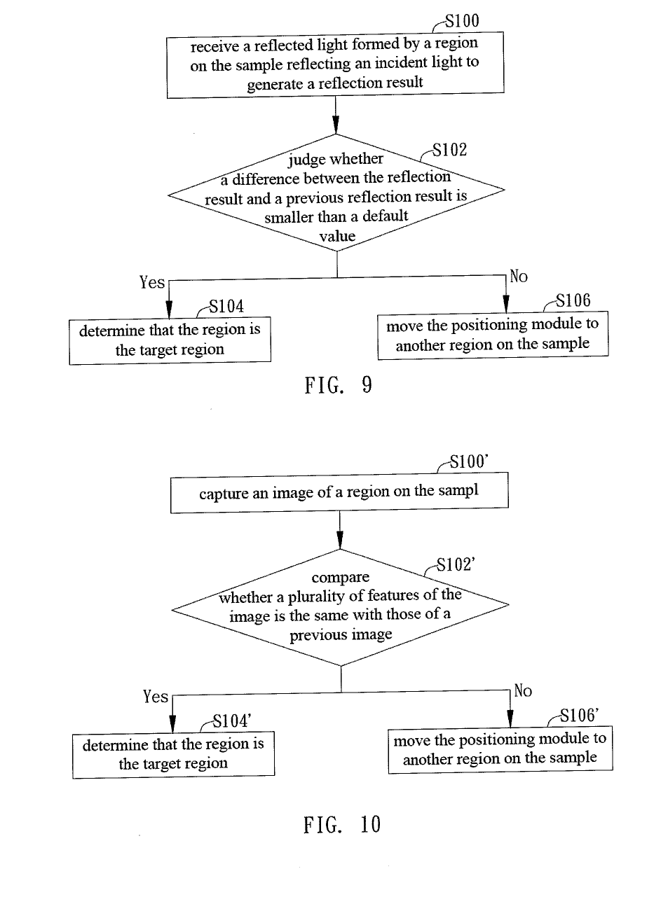

Taking the light emission/reflection positioning mechanism for example, as shown in FIG. 9, the step S10 of the method can include steps S100˜S106. At first, the method performs step S100 to receive a reflected light formed by a region on the sample reflecting an incident light to generate a reflection result. Then, the method performs step S102 to judge whether a difference between the reflection result and a previous reflection result is smaller than a default value. If the judged result of the step S102 is yes, the method will perform step S104 to determine that the region is the target region. If the judged result of the step S102 is no, the method will perform step S106 to move the positioning module to another region on the sample.

Taking the image comparing and positioning mechanism for example, as shown in FIG. 10, the step S10 of the method can include steps S100′˜S106′. At first, the method performs step S100′ to capture an image of a region on the sample. Then, the method performs step S102′ to compare whether a plurality of features of the image is the same with those of a previous image. If the judged result of the step S102′ is yes, the method will perform step S104′ to determine that the region is the target region. If the judged result of the step S102′ is no, the method will perform step S106′ to move the positioning module to another region on the sample.

In practical applications, the optical sensing module can use the optical interference technology (e.g., OCT technology) to perform the optical sensing on a tissue under the target region of the sample to obtain the optical information data related to a longitudinal profile of the tissue. The optical sensing module includes at least one optical component, a replaceable adapter interface, and a contact end replacing component.

Compared to prior arts, the skin optical diagnosing apparatus and operating method thereof of the invention can accurately confirm the target test position on the sample is the same with the previous test position through optical positioning method before performing optical sensing on the skin or similar tissues. Therefore, even though the skin optical diagnosing apparatus performs several times of optical diagnosing on human body skin, the positions in every time of optical diagnosing will be the same, so that the doctor can easily trace and compare the lesions of the patient to effectively enhance the quality and the efficiency of medical treatment. In addition, the optical sensing module of the skin optical diagnosing apparatus in this invention can be designed to be single component type or two kits combination type, and can include a replaceable adapter interface and a contact end replacing component, therefore, it has advantages of easy to upgrade, flexibly designed, avoiding pollution, and proving personal usage. If the optical sensing module is designed to be fixed type, it can cooperate with special rulers to perform more detailed and accurate positioning and comparing, and the optical sensing module can be also integrated with the positioning module in the same structure to perform large-area scanning test, so that the data of large-area preliminary observation, small-area detailed test, and test path record can be established respectively to help the doctor to continuously observe and trance the state of the patient.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A skin optical diagnosing apparatus, comprising:
a positioning module, for selecting a target region on a sample;
an optical sensing module, coupled to the positioning module, for performing an optical sensing on the target region to obtain an optical information data related to the target region;
a processing module, coupled to the optical sensing module, for processing the optical information data to generate an optical diagnosed result; and
a display module, coupled to the processing module, for displaying the optical diagnosed result.

2. The skin optical diagnosing apparatus of claim 1, wherein the optical sensing module uses an optical interference technology to perform the optical sensing on a tissue under the target region of the sample to obtain the optical information data related to a longitudinal profile of the tissue.

3. The skin optical diagnosing apparatus of claim 1, wherein the optical sensing module comprises at least one optical component, a replaceable adapter interface, and a contact end replacing component.

4. The skin optical diagnosing apparatus of claim 1, wherein the positioning module comprises:
a light receiving unit, for receiving a reflected light formed by a region on the sample reflecting an incident light to generate a reflection result; and
a judging unit, coupled to the light receiving unit, for judging whether a difference between the reflection result and a previous reflection result is smaller than a default value, if the judged result of the judging unit is yes,
the judging unit determines that the region is the target region.

5. The skin optical diagnosing apparatus of claim 1, wherein the positioning module comprises:
an image capturing unit, for capturing an image of a region on the sample; and
an image comparing unit, coupled to the image capturing unit, for comparing whether a plurality of features of the image is the same with those of a previous image, if the compared result of the image comparing unit is yes, the image comparing unit determines that the region is the target region.

6. A skin optical diagnosing apparatus operating method, applied to a skin optical diagnosing apparatus, the skin optical diagnosing apparatus comprising a positioning module, an optical sensing module, a processing module, and a display module, the skin optical diagnosing apparatus operating method comprising steps of:
(a) the positioning module selecting a target region on a sample;
(b) the optical sensing module performing an optical sensing on the target region to obtain an optical information data related to the target region;
(c) the processing module processing the optical information data to generate an optical diagnosed result; and
(d) the display module displaying the optical diagnosed result.

7. The skin optical diagnosing apparatus operating method of claim 6, wherein in the step (b), the optical sensing module uses an optical interference technology to perform the optical sensing on a tissue under the target region of the sample to obtain the optical information data related to a longitudinal profile of the tissue.

8. The skin optical diagnosing apparatus operating method of claim 6, wherein the optical sensing module comprises at least one optical component, a replaceable adapter interface, and a contact end replacing component.

9. The skin optical diagnosing apparatus operating method of claim 6, wherein the step (a) comprises steps of:
(a1) receiving a reflected light formed by a region on the sample reflecting an incident light to generate a reflection result;
(a2) judging whether a difference between the reflection result and a previous reflection result is smaller than a default value; and
(a3) if the judged result of the step (a2) is yes, determining that the region is the target region.

10. The skin optical diagnosing apparatus operating method of claim 6, wherein the step (a) comprises steps of:
(a1′) capturing an image of a region on the sample;
(a2′) comparing whether a plurality of features of the image is the same with those of a previous image; and
(a3′) if the compared result of the step (a2′) is yes, determining that the region is the target region.
